(19) United States
(12) Patent Application Publication
(10) Pub. No.: US 2008/0261234 A1
(43) Pub. Date: Oct. 23, 2008

(54) Peptides and their use in assays for cardiovascular disease

(76) Inventors: Outi Narvanen, Kuopio (FI); Seppo Yla-Herttuala, Kuopio (FI)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 11/800,148

(22) Filed: May 4, 2007

Related U.S. Application Data
(63) Continuation of application No. 10/333,313, filed on Jul. 2, 2003, now abandoned.

(30) Foreign Application Priority Data
Jul. 18, 2000 (GB) 0017641.2

(51) Int. Cl. G01N 33/53

(52) U.S. Cl. 435/7.1; 436/501

(57) ABSTRACT

A peptide having affinity for oxidised low density lipoprotein, in cyclised or multimeric form is useful in an enzyme immunosolvent assay, to detect oxLDL which is a marker of coronary heart disease.

### CROSS-REFERENCE TO A RELATED APPLICATION

This application is a continuation application of application Ser. No. 10/333,313, filed Jan. 15, 2003, now abandoned; which is a National Stage Application of International Application Number PCT/GB2001/003212, filed Jul. 18, 2001; which claims priority to Great Britain Application No. 0017641.2, filed Jul. 18, 2000, all of which are herein incorporated in their entirety.

### FIELD OF THE INVENTION

This invention relates to the preparation of peptides for use in assays for evaluating the risk of coronary heart diseases and other cardiovascular diseases.

### BACKGROUND TO THE INVENTION

Coronary heart disease (CHD) is the leading cause of death in European countries. In most cases, the basic cause of CHD is atherosclerosis. Currently, the risk of atherosclerosis is evaluated by measuring the amount of total cholesterol, low density lipoprotein (LDL) and high density lipoprotein (HDL). However, these tests do not predict the disease in approximately one third of the patients; see Yla-Herttuala, Current Opinion Lipidol. (1998) 9: 337-344. There is therefore a need to develop a better assay to predict the risk for CHD.

Oxidized low density lipoprotein (oxLDL) has been shown to be a risk factor in atherosclerosis, but it has not been possible to measure oxLDL directly in plasma because its half-life in circulation is short. Recent studies have therefore focused on different indirect measurements to define the extent of LDL oxidation.

OxLDL plays an important role in atherogenesis. It has been detected in atherosclerotic lesions, is cytotoxic to various cell types and chemotactic for blood monocytes. In addition, oxLDL is immunogenic, and atherosclerotic lesions contain immunoglobulins that recognize oxLDL; autoantibodies against oxLDL are present in human and rabbit sera. The best way to analyze oxLDL appears to be the measurement of autoantibodies against oxLDL, as suggested by Yla-Herttuala, supra.

Apolipoprotein B-100 (apoB-100) is the major protein constituent in LDL. The human cDNA and amino-acid sequences are reported by Chen et al., J. Biol. Chem. (1986) 261: 12912-12921.

During oxidation of LDL, both the protein and the lipid portion of the particle can be modified. Malondialdehyde (MDA) and 4-hydroxynonenal (4-HNE) are the main reactive aldehydes formed during LDL oxidation, as reported by Esterbauer et al., Free Radical Biol. Med. (1992) 13:341-390; each can further react with lysine residues of apoB-100. These poorly characterized oxidation specific epitopes are recognized by the autoantibodies. More recently, it has been suggested by Palinski et al., J. Clin. Invest. (1996) 98: 800-814, that oxidized phospholipids are epitopes for autoantibodies. In addition, it has been reported that healthy individuals produce antibodies against lysophosphatidylcholine, which is a major component of oxLDL.

Anti-oxLDL autoantibodies may predict progression of carotid atherosclerosis, coronary atherosclerosis and myocardial infarction. Elevated levels of autoantibodies have also been found in systemic lupus erythematosus (SLE), pre-eclampsia, chronic periaortitis, non-insulin-dependent diabetes mellitus and in endothelial dysfunction.

Autoantibodies against oxLDL have been measured using very different immunoassays (EIA or RIA), and no standard method or reference material is available for the standardization of the assays. LDL used in previous tests has been purified from human plasma and is usually oxidized by incubation with copper ions or by conjugation with MDA. Copper-oxidized LDL contains a mixture of oxidation-specific epitopes, and therefore the oxidation process must be standardized carefully to produce homogeneous antigen. Human plasma LDL-based antigens are also inherently unstable and are not suitable for the production of commercial test kits. Therefore, there is a need to produce an assay for CHD that can be standardised and which makes use of reagents which are stable and give reproducible results.

### SUMMARY OF THE INVENTION

The present invention is based on the realisation that suitable peptides, e.g. modified with a reactive aldehyde, are stable and can be used as antigens in an immunoassay for CHD. More generally, a novel peptide has affinity for oxidised low density lipoprotein, in cyclised or multimeric form.

According to one aspect of the invention, a peptide of the invention is used in an immunoassay to determine the presence and, optionally, the amount of antibodies, in a sample, having affinity for oxidised low density lipoprotein.

According to a second aspect, a method for measuring the amount of autoantibodies for oxidised low density lipoprotein in a sample, comprises:

(i) contacting the sample with immobilised, derivatised peptides as defined above, under conditions which permit the autoantibodies to bind to the peptides; and

(ii) determining the amount of binding.

The amount of binding can be measured directly and will correlate to the amount of oxidised LDL in a sample. The amount of antibodies can be expressed as the ratio of antibody binding between oxidised LDL and native LDL.

The use of the derivatised peptides ensures that the immunoassay uses a stable antigen which provides reproducible results.

According to a third aspect, a kit for measuring autoantibodies of oxidised LDL, comprises a multicontainer unit having:

(i) a composition comprising derivatised peptides as defined above; and

(ii) reagents necessary to carry out an immunoabsorption assay.

The present invention provides reagents that can be synthesised easily without the need to isolate proteins from a patient's blood. The peptides do not have the short half-life associated with the proteins used in conventional assays for CHD and therefore can be manufactured commercially for use in diagnostic kits.

### BRIEF DESCRIPTION OF THE DRAWINGS

FIG. 1 shows titration curves for 4 peptide antigens tested with one patient sample.

FIG. 2A shows results of an inhibition test of cyclic peptide p244cyc on 8 patient samples.

FIG. 2B shows results of an inhibition test of linear peptide p244 on 24 patient samples.

FIG. 3 shows results of an inhibition test of p63 peptide on 22 patient samples.

FIG. 4 shows a comparison of titers of autoantibodies against peptides p244 and p244-MDA to four groups (quartiles) of titers of autoantibodies against oxLDL.

FIG. 5 shows a comparison of the amount of antibodies against peptides p244 and p244-MDS to angina pectoris symptoms classified according to the New York Heart Association (NYHA) heart disease classification.

FIG. 6 shows the total sum of coronary stenosis compared with NYHA classification.

### DESCRIPTION OF THE INVENTION

The present invention relies on the production of peptides which are preferably derived from apoB-100 protein, or which preferably have an amino acid sequence which forms a structure similar to that of the epitopes on apoB-100 protein. The peptides are therefore able to undergo specific interaction with autoantibodies which have affinity for oxidised LDL. The term “specific interaction” refers to the recognition of the autoantibodies for the peptide (antigen). The peptides may elicit antibody binding with an affinity constant of greater than 105 mmol, preferably greater than 10⁷ I/mol and more preferably greater than 10⁸ I/mol.

In principle, any peptide sequence of approximately greater than 10 amino acids may be used in the present invention provided that it acts as a ligand for the autoantibodies. The peptide may be derived from a natural source of apoB-100 or may be a synthetic peptide based on the known protein sequence for apoB-100. Methods to isolate peptides from apoB-100 or to synthesis peptides, will be apparent to the skilled person.

The peptides are derivatised with a reactive aldehyde on any suitable amino acid. Preferably, the peptides are derivatised on an arginine, histidine or lysine residue. Methods for derivatising the peptides, in addition to those disclosed herein, will also be apparent to the skilled person.

The reactive aldehyde used to derivatise the peptides may be maiondialdehyde or hydroxynonenal. Others will be apparent to the skilled person.

The size of the peptides is sufficient for recognition by the autoantibodies. Preferably, the peptides are 10-40 amino acids in size, more preferably 15-30 amino acids. The amino acid sequence of the peptides is preferably greater than 80%, preferably greater than 90%, and most preferably greater than 95% identical to a region on the native apoB-100 protein.

The peptides of the present invention may be used in a diagnostic assay together with other reagents capable of eliciting an antibody reaction. For example, phosphatidyl ethanolamine can be derivatised with MDA and, when used with the peptides of the invention, is capable of acting as an epitope for some autoantibodies.

The new peptide-based EIA assay could be used as a test kit for the evaluation and follow-up of patients with cardiovascular diseases and several other disorders, such as periaortitis, pre-eclampsia, non-insulin-dependent diabetes and endothelial dysfunction.

When used in the immunoassay, it is preferable that the peptides are immobilised on a solid support, as this enables subsequent washing steps to be carried out easily. Methods to carry out immunoassays will be apparent to the skilled person.

The following Examples illustrate the invention.

### EXAMPLE 1

Various native and modified peptides, derived from the amino acid sequence of apoB-100 (Chen et al., supra), were tested as antigens suitable for use in EIA. The peptides were modified with MDA to produce similar oxidation specific epitopes as in oxLDL. The results of peptide EIA are compared to the results of oxLDL EIA which was optimized using copper-oxidized LDL as antigen; see Närvänen et al., Free Radical Biology & Medicine, in press.

Peptides were synthesized by using solid-phase peptide synthesis technology and Fmoc chemistry and purified with HPLC using a C₁₈ column. The molecular weights of the synthesized peptides were identified by using a MALDI-TOF mass spectrometer. Peptide sequences either derived from or unrelated to the amino acid sequence of apoB-100 are shown in Table 1.

TABLE 1
Sequences of the synthetic peptides

| | | | |
|---|---|---|---|
| | | Number | Number |
| | | of | of |
| | | amino | lysines |
| Peptide | Sequence | acids | (═K) |

| | | | |
|---|---|---|---|
| p62 | DIVAHLLSSSSSVIDALQYKLE | 35 | 3 |
| SEQ ID NO. 1 | GTTRLTRKRGLK | | |
| p63 | LSVKAQYKKNKHRHSITNPL | 20 | 4 |
| SEQ ID NO. 2 | | | |
| p64 | STTVMNPYMKLAPGELTIIL | 20 | 1 |
| SEQ ID NO. 3 | | | |
| p65 | KKKKKKKKKKKKKKKKKKKKKK | 35 | 35 |
| SEQ ID NO. 4 | KKKKKKKKKKKKK | | |

Modification of the Peptide with MDA

MDA was coupled to the peptide via the primary amines of lysines by using a modification of the method of Palinski et al., Arteriosclerosis (1990) 10: 325-335. MDA was made freshly from malondialdehyde-bis(dimethyl acetal) by acid hydrolysis. 10 moles of MDA was then coupled to 1 mole of peptide by stirring the mixture for 3 hours at 37° C. The efficiency of coupling was checked by ninhydrin reaction which reveals the presence of primary amines.

Serum and plasma samples for peptide EIA tests were collected from ongoing studies including samples from patients with suspected coronary heart disease and from healthy controls. These samples were stored in aliquots at −20° C.

### Autoantibodies Against Peptides and Peptide-MDA Complexes (Peptide EIA)

Native and MDA-modified peptides were tested always on the same plate. One half of the plate was coated with the native peptide (20 μg/ml) and the other half was coated with the MDA-modified peptide [(20 μg/ml) 100 μl/well in 100 mmol/l bicarbonate buffer (pH 9.5)]. Coated plates were incubated overnight at room temperature (RT) and then washed with an automatic washer (Wellwash 4 MK II, Labsystems Oy) three times with phosphate buffered saline (PBS) containing 0.05% Tween 20. Plates were blocked with PBS containing 1% human serum albumin (HSA) (150 μl/well) for 1 h at RT and washed as above. Serum samples were diluted 1:20 in PBS containing 0.2% HSA and 0.05% Tween 20 and pipetted 100 μl/well. Plates were incubated for 2 h at RT and washed as above. HRP-conjugated anti-human IgG (diluted 1:20 000 in the sample buffer) was added (100 μl) to each well and incubated for 1 h at RT. After washing, colour was developed by adding the peroxidase substrate (tetramethylbenzidine (TMB) as a chromagen, 100 μl/well) and incubating the plates for 30 min at room temperature in the dark. The reaction was stopped with 0.5 mol/l H₂SO₄ (100 μl/well) and absorbances were measured at 450 nm (Multiskan microplate reader, Labsystems Oy). The results are shown in Table 2 and are expressed as the absorbance measurement obtained for the native and the modified peptides or as the ratio between antibody binding to the native and the modified peptides after subtracting the blank control.

TABLE 2
Reactivity of autoantibodies

| | |
|---|---|
| OxLDL EIA | PEPTIDE EIA |

A. Healthy controls (n = 17).

| | | | |
|---|---|---|---|
| oxLDL:natLDL | 1.886 ± 0.689 | MDAp63:natp63 ratio | 2.592 ± 0.751 |
| ratio | | | |
| oxLDL | 0.591 ± 0.221 | MDAp63 | 0.497 ± 0.184 |
| natLDL | 0.351 ± 0.172 | natp63 | 0.216 ± 0.142 |

B. Patients (n = 19).

| | | | |
|---|---|---|---|
| oxLDL:natLDL | 2.535 ± 1.430 | MDAp63:natp63 ratio | 2.866 ± 0.924 |
| ratio | | | |
| oxLDL | 0.600 ± 0.287 | MDAp63 | 0.418 ± 0.141 |
| natLDL | 0.258 ± 0.170 | natp63 | 0.159 ± 0.079 |

Based on the ratio values, the amount of autoantibodies was higher in patient samples than in controls when tested with the peptide EIA and the human LDL-based oxLDL EIA. The reactions of autoantibodies using the native and modified (peptide) antigens are similar in oxLDL-EIA and peptide-EIA because the modified antigens (oxLDL and MDAp63) are recognized better than the native antigens (natLDL and natp63), i.e. there is greater specificity for the modified antigens. The results for the peptide-EIA are significantly better than for the oxLDL-EIA.

To find out whether autoantibodies react with the peptide, with MDA or both when peptide-MDA complexes are used as antigen, a microtiter plate was coated with natp63, MDA and MDAp63 antigen and the immunoassays performed. MDA antigen was prepared as MDAp63 antigen but without peptide and MDA was diluted for coating in the same way as MDAp63. The reaction of autoantibodies was higher with MDAp63 than with natp63 and MDA, in both control (0.533±0.077, 0.282±0.100 and 0.300±0.019, respectively, mean ±SD n=23) and patient samples (0.431±0.040, 0.222±0.041 and 0.338±0.000, respectively, n=16), indicating that both peptide and MDA are needed for optimal recognition.

Spearman correlation coefficients were calculated between antibodies against natp63, MDA and MDAp63. Anti-MDA and anti-MDAp63 antibodies correlated in control and patient samples (r=0.581 p=0.004 and r=0.582 p=0.018, respectively), but anti-natp63 and anti-MDA antibodies did not correlate (r=0.297 p=0.169 and r=0.112 p=0.68, respectively). This shows that different antibodies react with MDA and natp63 and is evidence that the peptide-MDA complex is the optimal antigen.

### EXAMPLE 2

Six different forms of peptide p63 were tested, to find out what is the best modification of the peptide for EIA:

linear p63 (p63)
p63 modified with MDA (p63-MDA)
p63 modified with MDA and phosphatidylethanolamine (p63-PEA-MDA)
a linear form of p63, which contains two extra amino acids (glycine and cysteine) at both ends of the peptide (p244); see SEQ ID NO. 5
cyclic p244 (p244cyc)
cyclic p63 modified with MDA (p244cyc-MDA)

The amount of autoantibodies was highest in patient samples when the linear peptide p244 was used as antigen (Table 3). Antibody titers against cyclic peptide 244cyc were also slightly higher than against p63-MDA. In this study autoantibodies reacted most with peptides without lipids.

### Specificity Studies

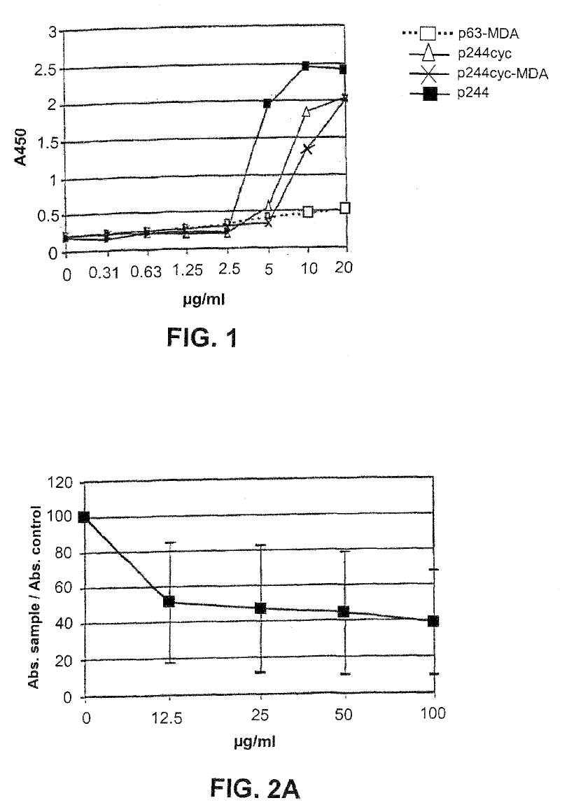

The specificity of different antigens was tested by titration of peptides. FIG. 1 shows titration curves for 4 peptide antigens tested with one patient sample. In addition, the specificity of antibodies against cyclic and linear peptides, p244cyc and p244, was tested by adding 0.5 M sodium chloride to sample diluent. The titer of patient samples against p244, p244cyc and p63-MDA was 19%, 38% and 47% lower (mean of 20 patient samples), respectively, when salt was added to sample diluent. It appears that antibodies against p244 have the best specificity.

### Inhibition Studies

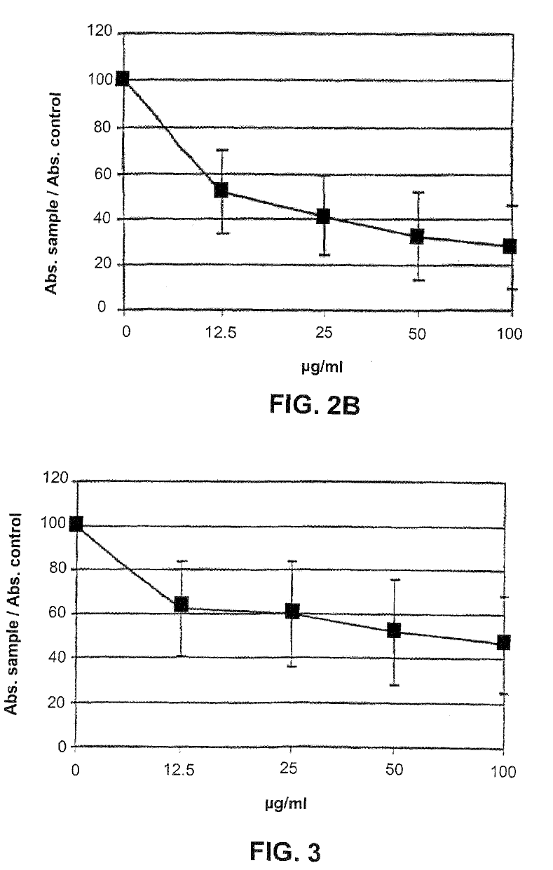

The specificity of cyclic (p244cyc) and the corresponding linear peptide (p244) were tested in inhibition tests by adding increasing concentrations of peptides to sample diluent. P244cyc peptide inhibited with the highest inhibitor concentration (100 μg/ml), the average being 61% of the binding of 8 patient samples (FIG. 2A); 3 samples were inhibited by less than 50%. P244 inhibited with the highest inhibitor concentration (100 μg/ml), the average being 70% of the binding of 24 patient samples (FIG. 2B). 3 samples were inhibited less than 50%. P244 seemed to be more specific than p244cyc peptide. Finally, it was confirmed that the epitope of p244 is similar to p63 by coating plate with p244 and inhibiting the binding of patient samples with p63. P63 inhibited with the highest inhibitor concentration (100 μg/ml), the average being 53% of the binding of 22 patient samples (FIG. 3). 9 samples were inhibited less than 50%.

Modification of p244

As a summary of previous results it appears that patient samples are best recognized by the peptide p244. Therefore, modified p244 was modified with MDA and p244 and p244-MDA were both used as antigen in EIA. The titers of patient samples (mean_SD) against p244 and p244-MDA were slightly higher than titers of control samples (Table 5).

Evaluation of Peptide EIA

205 samples of patients with symptoms of CHD were analysed. The patients were angiographically tested and classified as 0-, 1-, 2-, or 3-vessel disease. Peptides p244 and p244-MDA were used as antigen in peptide EIA, and the results compared to human-LDL-based oxLDL EIA.

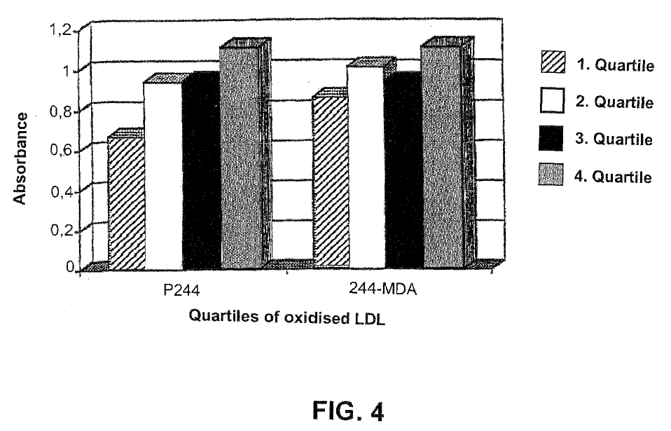

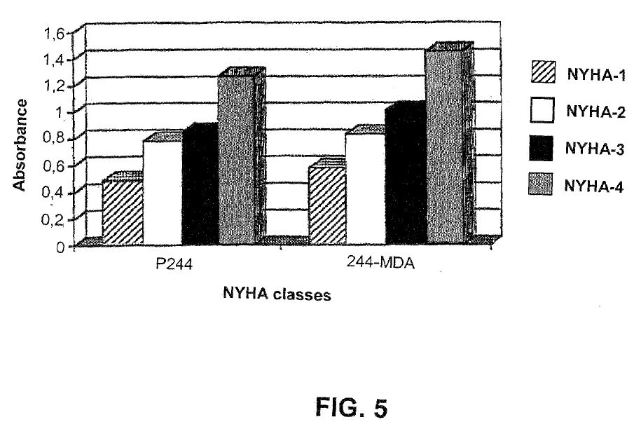

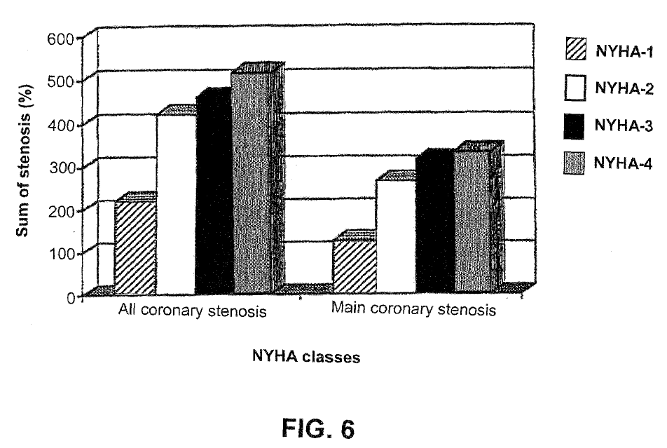

The titers of autoantibodies against p244-MDA were highest in those patients with 3-vessel disease compared to 0-, 1-, or 2-vessel disease (ANOVA p=0.0268). The titers of autoantibodies against human-LDL-based oxLDL (n=185) correlated with the titers against p244 (r=0.227 p=0.0019) and with the titers against p244-MDA (p=0.217 p=0.003). The correlation was confirmed when the titers of autoantibodies against peptides p244 and p244-MDA were compared to the titers of autoantibodies against human-LDL-based oxLDL classified to four groups based on anti-oxLDL titer (1. quartile 0.072-0.22 (n=37), 2. quartile 0.22-0.36 (n=44), 3. quartile 0.36-0.63 (n=57), and 4. quartile 0.63-2.757 (n=68)) (FIG. 4). In addition, we compared the amount of antibodies against p244 and p244-MDA to angina pectoris symptoms classified according to the New York Heart Association (NYHA) heart disease classification (NYHA-1 n=5, NYHA-2 n=12, NYHA-3 n=27, and NYHA-4 n=14). We found that antibody titers increased when angina pectoris symptoms increased (FIG. 5). The total sum of coronary stenosis compared with NYHA classification (NYHA-1 n=14, NYHA-2 n=26, NYHA-3 n=57, and NYHA-4 n=29) showed more serious symptoms with the progression of coronary disease (FIG. 6). This result revealed that the total sum of coronary stenosis examined using coronary angiography is correlated as well as the antibodies with the seriousness of angina pectoris symptoms.

TABLE 3
The amount of autoantibodies against six
different forms of p63 peptide antigen

| Peptide | | P63- | P63-PEA- | | P244cyc- | |
|---|---|---|---|---|---|---|
| antigen | P63 | MDA | MDA | P244cyc | MDA | P244 |
| Patients | 0.389_{—} | 0.601_{—} | 0.406_{—} | 0.734_{—} | 0.521_{—} | 0.995_{—} |
| (n = 20) | 0.353 | 0.192 | 0.215 | 0.520 | 0.504 | 0.570 |

Values are mean absorbances_SD after subtracting the blank.

TABLE 4
The amount of autoantibodies against
p244 and MDA modified p244 peptide

| Peptide antigen | P244 | P244-MDA |
|---|---|---|
| Controls (n = 23) | 0.870_0.371 | 0.956_0.307 |
| Patients (n = 20) | 1.074_0.610 | 1.296_0.544 |

Values are mean absorbances_SD after subtracting the blank.

We claim:

1. A method for detecting antibodies to oxidized low density lipoprotein in a sample, wherein said method comprises:
i) contacting the sample with a peptide that has up to 40 amino acids and that comprises SEQ ID NO. 2, under conditions that permit antibodies to bind to the peptide; and
ii) detecting whether binding occurs between any antibodies present and the peptide,
wherein such binding indicates that antibodies to oxidized low density lipoprotein are present in the sample.

2. The method according to claim 1, wherein the peptide interacts with the antibody with an affinity constant of greater than 10⁵ I/mol.

3. A method for measuring an amount of antibodies to oxidized low density lipoprotein in a sample, comprising:
i) contacting the sample with a peptide wherein the peptide has up to 40 amino acids and comprises SEQ ID NO. 2, under conditions which permit antibodies to bind to the peptide; and
ii) determining the amount of binding.
wherein the amount of binding of the peptide to antibodies in the sample is correlated to the amount of antibodies to oxidized low density lipoprotein in the sample.

4. The method according to claim 3, wherein the sample is a serum or plasma sample from a patient.

5. A kit suitable for measuring antibodies to oxidized low density lipoprotein in a sample, comprising a unit including containers respectively containing:
i) a peptide wherein the peptide has up to 40 amino acids and comprises SEQ ID NO. 2; and
ii) reagents necessary to carry out an immunoadsorbence assay.

6. An isolated peptide wherein the peptide has up to 40 amino acids and comprises SEQ ID NO. 2.


SEQUENCE LISTING (5 sequences)

SEQ ID NO: 1 - protein, 34 aa, Artificial Sequence
  oligopeptide
DIVAHLLSSSSSVIDALQYKLEGTTRLTRKRGLK

SEQ ID NO: 2 - protein, 20 aa, Artificial Sequence
  oligopeptide
LSVKAQYKKNKHRHSITNPL

SEQ ID NO: 3 - protein, 20 aa, Artificial Sequence
  oligopeptide
STTVMNPYMKLAPGELTIIL

SEQ ID NO: 4 - protein, 35 aa, Artificial Sequence
  oligopeptide
KKKKKKKKKKKKKKKKKKKKKKKKKKKKKKKKKKK

SEQ ID NO: 5 - protein, 24 aa, Artificial Sequence
  oligopeptide
CGLSVKAQYKKNKHRHSITNPLGC